(19) United States
(12) Patent Application Publication
(10) Pub. No.: US 2009/0275831 A1
(43) Pub. Date: Nov. 5, 2009

(54) IMAGE REGISTRATION AND METHODS FOR COMPENSATING INTRAOPERATIVE MOTION IN IMAGE-GUIDED INTERVENTIONAL PROCEDURES

(75) Inventors: Christopher Hall, Hopewell Junction, NY (US); Hui M. Jiang, Issaquah, WA (US); Gary A. Schwartz, Seattle, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/521,066

(22) PCT Filed: Dec. 27, 2007
(86) PCT No.: PCT/IB07/55319

§ 371 Date: Jun. 24, 2009

Related U.S. Application Data
(60) Provisional application No. 60/882,669, filed on Dec. 29, 2006.

(51) Int. Cl. A61B 8/00; G06K 9/00

(52) U.S. Cl. 600/437; 382/128

(57) ABSTRACT

The invention provides methods and systems for guiding an interventional medical procedure using ultrasound imaging. Using improved image fusion techniques, the invention provides an improved method for the treatment of a flexible target volume and/or flexible surrounding structures.

The technical field is methods and systems for ultrasound guidance in an interventional medical procedure.

An interventional medical procedure typically involves inserting a small biomedical device (e.g. a needle or a catheter) into a patient body at a target anatomic position for diagnostic or therapeutic purposes. Images from various imaging modalities are used for guiding insertion and/or adjusting placement of the device. One such modality is ultrasound grayscale imaging, which provides a static image and/or an image in real time, is non-invasive, and operates at low cost. An ultrasound scanner also effectively visualizes the interventional device and is easily used in conjunction with the device.

However, it has been difficult to use an ultrasound grayscale modality to image certain types of tissue, for instance, those that have an inconsistent or unspecific acoustic signature relative to surrounding healthy tissue. For instance, hepatocellular carcinomas have been difficult to detect because they are hypoechoic, hyperechoic, or isoechoic with the surrounding healthy liver parenchyma. Therefore, successful ultrasound guidance of interventional treatment of this type and similar types of malignant tissue has been difficult. Therefore, information obtained from more sensitive modalities (e.g. computed tomography (CT), contrast enhanced ultrasound (CEUS), or magnetic resonance imaging (MRI)) has been used for producing preoperative images of a target volume, while still using ultrasound grayscale to image the interventional device. A co-registration technique then combines a preoperative image with a real time ultrasound image. Combining target volume location from the preoperative image with device location from the ultrasound image adds to a physician's confidence and accuracy in the placement of the interventional device.

Current co-registration techniques involve registering images from different modalities (e.g. CT and ultrasound images). Cross-modality co-registration is often expensive and requires a long computation time. Co-registration between CEUS and grayscale ultrasound has also been difficult because a CEUS image used to detect the target volume is time variant, whereas a grayscale ultrasound image used to monitor the interventional device is not time variant.

Further, most current co-registration techniques assume that target organs and surrounding structures are static solid objects and ignore organ motion or deformation during an interventional procedure. However, organ (e.g. respiratory and/or cardiac) motion or patient general body motion are often non-negligible during treatment. Typical displacements on the order of 10-30 mm in abdominal targets have been observed (Rohlfing T. Maurer C R Jr. O'dell W G. Zhong J. Medical Physics. 31(3):427-32, 2004 Mar.). Those displacements produce a poor estimation of the correct position of a target volume and therefore result in inaccurate treatment.

In image-guided neurosurgery, the problem of motion (known as “brain-shift”) compensation of the preoperative image or dataset has been addressed by studies that use perioperative ultrasound for displacement estimation. In a recent study by Lunn et al., for example, stainless-still beads were implanted in pigs' brains (Lunn, K. E., Paulsen, K. D., Roberts, D. W., Kennedy, F. E., Hartov, A., West, J. D. Medical Imaging, IEEE Transactions on, 22(11), pp. 1358-1368, November 2003). Using the beads as markers, the brains were imaged in a three-dimensional preoperative CT scan, and then tracked by ultrasound. This tracking allowed retrieving the translation vector of the brain-shift motion model. The preoperative dataset was then corrected by inverting the inferred translation vector. The main disadvantages of this method are the invasive insertion of markers, and the assumption of translation-only motion, which ignores the deformation that occurs within target structures that are soft (for instance, brain or liver) or structures that are surrounded by soft and/or moving tissue (for instance, heart or diaphragm). There is a need for improved methods of correcting imaging data for target movement.

Accordingly, a featured embodiment of the invention provided herein is a method for computing non-invasively a velocity vector field of a flexible target volume within a bodily cavity, including: generating a preoperative image of a region surrounding the target volume using a preoperative imaging modality, wherein the region comprises the target volume and wherein the modality is not grayscale ultrasound, and producing a initial target volume calculation; generating an ultrasound image of a region surrounding the target volume using an ultrasound imaging modality, wherein the region comprises the target volume, spatially aligning the ultrasound image with the preoperative image using an image co-registration technique, thereby providing an updated target volume calculation, and combining the ultrasound image with the preoperative image using an overlay technique; and computing the velocity vector field of the target volume, wherein computing the field is non-invasive and is adjusted to a flexibility value of the target volume and surrounding tissue.

In a related embodiment of the method, the preoperative image and/or preoperative modality is at least one of the following types: magnetic resonance, computed tomography, contrast enhanced ultrasound, and the like.

In another related embodiment, at least one of the initial target volume calculation and the updated target volume calculation further comprises at least one of the following target volume parameters: a location, an extent, and a shape of the target volume.

In yet another related embodiment, the ultrasonic image is a two-dimensional image or a three-dimensional image. In a related embodiment, the ultrasonic image is used to estimate the velocity vector field of the target volume by comparing successive frames of ultrasound intensity data.

In a related embodiment of the above method, computing the velocity vector field involves computing a displacement field. In another related embodiment, computing the velocity vector field and/or displacement field includes calculating at least one of the following target volume parameters: rotation, translation, and deformation of the target volume.

A related embodiment includes reducing computation time by at least one of the following steps: generating a single preoperative image, using a single image co-registration, and using a single imaging modality to compute the velocity vector field and/or displacement field.

Another featured embodiment of the invention provided herein is a method for guiding an interventional medical procedure for diagnosis or therapy of a flexible target volume, including: using a velocity vector field and/or displacement field of the target volume to modify in real time a target volume calculation, in which computing the field is non-invasive and adjusted to a flexibility value of the target volume and surrounding tissue; generating at least one ultrasonic image of an interventional device in real time; and using a real time ultrasonic image of the interventional device and the ultrasonic target volume calculation to alter the placement of the interventional device, thereby guiding an interventional medical procedure for diagnosis or therapy of a flexible target volume.

In a related embodiment of the above method, the target volume calculation includes at least one of the following parameters: a location, an extent, and a shape of the target volume.

In another related embodiment, the ultrasonic image is a two-dimensional image or a three-dimensional image.

Another exemplary embodiment is a method for combining a plurality of types of medical images for guiding an interventional medical procedure. The method includes the following steps: generating a initial image of a region surrounding a target volume using an imaging modality, in which the region comprises the target volume and in which the modality is not grayscale ultrasound; generating a corresponding ultrasound index image; generating in real time an ultrasound image of the target volume; making an image-based co-registration between the ultrasound index image and a real time ultrasound image; and combining the initial image with the real time ultrasound image, using an overlay technique and/or the ultrasound index image.

In a related embodiment of the above method, the image or the imaging modality includes at least one of the following types: computed tomography, magnetic resonance imaging, contrast enhanced ultrasound, and the like.

In another related embodiment, the real time ultrasound image is generated during the interventional procedure.

Another exemplary embodiment is a system for guiding an interventional medical procedure using a plurality of imaging modalities. The system includes the following components: a preoperative imaging modality for generating a preoperative image and for producing a initial target volume calculation, in which the modality is not grayscale ultrasound; an ultrasound imaging modality for generating in real time an image of an interventional medical device and/or computing a velocity vector field and/or displacement field of the target volume, in which the field is used for generating an updated target volume calculation; and the interventional medical device for inserting into the target volume, in which the updated target volume calculation and the real time image of the interventional device are used to alter the placement of the interventional device.

In a related embodiment of the above method, the preoperative modality or preoperative image includes at least one of the following types: computed tomography, magnetic resonance imaging, contrast enhanced ultrasound, and the like.

In another related embodiment, the initial target volume calculation and/or the updated target volume calculation include at least one of the following target volume parameters: a location, an extent, and a shape of the target volume.

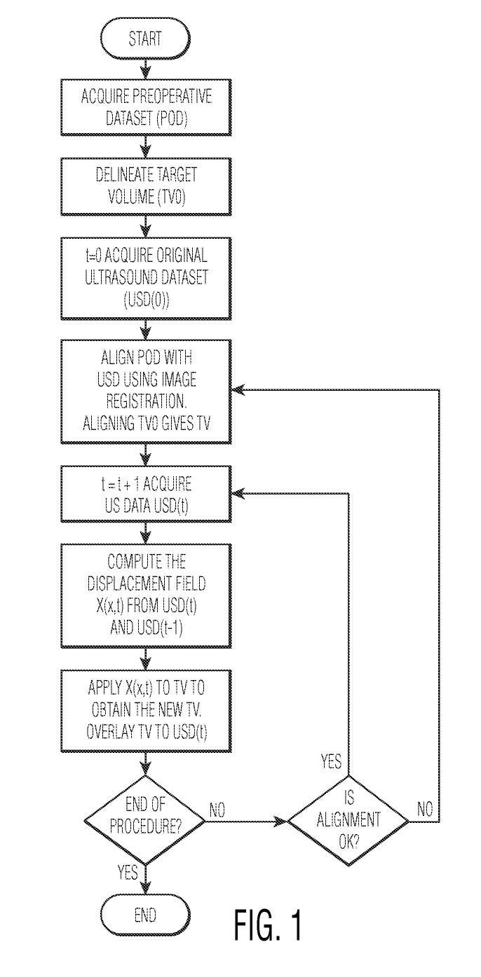

FIG. 1 is a flowchart showing guidance of an interventional medical procedure using imaging data.

An exemplary embodiment of the methods and systems provided herein is shown in FIG. 1. A preoperative dataset (identified as POD in FIG. 1) is calculated by an imaging modality (e.g. CT, MRI, and/or CEUS). This dataset is then used for generating an initial target volume calculation (identified as TVO in FIG. 1). An ultrasound dataset is then calculated, using an ultrasound imaging modality. The ultrasound dataset is then aligned with the preoperative dataset, using a co-registration technique. Aligning the preoperative dataset with the ultrasound dataset provides an updated target volume calculation (identified as TV in FIG. 1). Successive ultrasound datasets are then computed in real time and used to calculate a velocity vector field and/or displacement field of the target volume. The velocity vector field and/or displacement field provides a further updated target volume calculation. The updated target volume is then superimposed onto a real time ultrasound image of an interventional device, which improves the guidance and navigation of the device within a patient body.

An interventional medical procedure typically involves inserting a small biomedical device (e.g. a needle or catheter) into a patient body at a target anatomic position for diagnostic or therapeutic purposes. Examples of an interventional medical procedure include but are not limited to: radiofrequency ablation therapy, cryoablation, and microwave ablation.

Each image fusion technique is also useful for applications related and/or unrelated to guiding interventional medical procedures, for instance for non-invasive medical procedures or for instance non-medical procedures. Similarly, the method provided herein for calculating a velocity vector field and/or displacement field of a flexible target volume is also useful for applications related and/or unrelated to guiding interventional medical procedures, for instance for non-invasive medical procedures or for instance non-medical procedures.

The phrase “target volume,” as used herein, describes a physical three-dimensional region within a patient body which is or includes the intended site of interventional treatment. A target volume calculation includes an estimate of a size, shape, extent, and/or location within the patient body of the target volume.

A “flexible target volume,” as used herein, describes a target volume that has a flexibility value. A flexibility value describes an ability or propensity to bend, flex, distort, deform, or the like. A higher flexibility value corresponds to an increased ability or propensity to bend, flex, distort, deform, or the like.

A preoperative dataset is used to optimally detect and distinguish the target volume from surrounding parenchyma. A dataset, as used herein, refers to the data calculated by an imaging modality, and is used synonymously with the term “image.” In the methods and systems provided herein, CT, MRI, and/or CEUS modalities provide the preoperative dataset.

Ultrasound imaging (also referred to as medical sonography or ultrasonography) is a diagnostic medical imaging technique that uses sound waves that have a frequency greater than the upper limit of human hearing (the limit being about 20 kilohertz). Ultrasound imaging is used to visualize size, structure, and/or location of various internal organs and is also sometimes used to image pathological lesions. There are several types of ultrasound imaging, including grayscale ultrasound and CEUS. In general, a grayscale digital image is an image in which the value of each pixel is a single sample. Displayed images of this sort are typically composed of shades of gray, varying from black at the weakest intensity to white at the strongest, though in principle the samples could be displayed as shades of any color, or even coded with various colors for different intensities. Grayscale images are distinct from black-and-white images, which in the context of computer imaging are images with only two colors, black and white; grayscale images have many intermediate shades of gray in between the dichotomy of black and white. Unless otherwise specified, any reference to ultrasound provided herein, for instance, an ultrasound image or images, an ultrasound scanner or scanners, or an ultrasound modality or modalities refers to grayscale ultrasound.

The method provided herein uses ultrasound images for several purposes. Ultrasound images provide, in real time, a position of the interventional device. Ultrasound images are also used, in 2D and/or in 3D, to estimate the velocity field and/or displacement field of the target volume. A velocity vector field describes how a speed and a direction of motion of the target volume changes with time. A displacement field describes how a position of the target volume changes with time. The field is calculated by comparing ultrasound intensity values from successive ultrasound images. The velocity field and/or displacement field includes at least one of the following parameters: rotation, translation, and deformation of the target volume and/or surrounding tissues.

Although computation time increases with level of complexity of the velocity field and/or displacement field estimate, the computation time for the current method, which uses two ultrasound datasets, is considerably reduced compared to that in the prior art, in which computing the field involves image-based co-registration of images from different modalities.

Ultrasound is an effective modality for achieving motion estimation in high resolution. For example, the method provided herein uses block-matching techniques at a high frame rate, thereby obtaining resolution on the order of tenth of a millimeter in an axial direction (parallel to the axis of imaging).

In a typical block matching method, an image frame is divided into blocks of pixels (referred to herein as “blocks”). A standard block is rectangular in shape. A block matching algorithm is then employed to measure the similarity between successive images or portions of images on a pixel-by-pixel basis. “Successive images” are images obtained consecutively in time. For instance, five images are obtained per second; the second image is a successive image of the first image, the third image is a successive image of the second image, the fourth image is a successive image of the third image, and so forth. A block from a current frame is placed and moved around in the previous frame using a specific search strategy. A criterion is defined to determine how well the object block matches a corresponding block in the previous frame. The criterion includes one or more of the following: mean squared error, minimum absolute difference, sum of square differences, and sum of absolute difference. The purpose of a block matching technique is to calculate a motion vector for each block by computing the relative displacement of the block from one frame to the next.

Contrast-enhanced ultrasound (CEUS) describes the combination of use of ultrasound contrast agents with grayscale ultrasound imaging techniques. Ultrasound contrast agents are gas-filled microbubbles that are administered intravenously into systemic circulation. Microbubbles have a high degree of echogenicity, which is the ability of an object to reflect ultrasound waves. The echogenicity difference between the gas in the microbubbles and the soft tissue surroundings of the body is very great. Thus, ultrasonic imaging using microbubble contrast agents enhances the ultrasound backscatter, or reflection of the ultrasound waves, to produce a unique sonogram with increased contrast due to the high echogenicity difference. CEUS is used to image blood perfusion in organs, measure blood flow rate in the heart and other organs, and has other applications as well.

Computed tomography (CT) describes a medical imaging method that generates a three-dimensional image of an interior of an object from several two-dimensional X-ray images taken around a single axis of rotation. CT produces a volume of data which can be manipulated, through a process known as windowing, in order to demonstrate various structures based on how the structures block an x-ray beam. Modern scanners also allow a volume of data to be reformatted in various planes (as 2D images) or as a volumetric (3D) representation of a structure.

Magnetic resonance imaging (MRI), also referred to as magnetic resonance tomography (MRT) or nuclear magnetic resonance (NMR), describes a method used to visualize an interior of a living organism using powerful magnets and radio waves. MRI is primarily used to demonstrate pathological or other physiological alterations of living tissues and is a commonly used form of medical imaging. Unlike conventional radiography and CT imaging, which make use of potentially harmful radiation (x-rays), MRI imaging is based on the magnetic properties of atoms. A powerful magnet generates a magnetic field roughly 10,000 times stronger than the magnetic field of the earth. A very small percentage of hydrogen atoms within a body, e.g. a human body, will align with this field. Focused radio wave pulses are broadcast towards the aligned hydrogen atoms in a tissue; then, the tissue returns a signal. The subtle differences in that signal from various body tissues enables MRI to differentiate organs, and potentially contrast benign and malignant tissue. Any imaging plane (or slice) can be projected, stored in a computer, or printed on film. MRI is used to image through clothing and bones. However, certain types of metal in the area of interest can cause significant errors, called artifacts, in resulting images.

Image co-registration involves spatially aligning images using spatial coordinates, usually in three dimensions. In some embodiments, co-registration involves a manual image similarity assessment. In other embodiments, co-registration involves an image-based automated image similarity assessment. In some embodiments, co-registration involves an image-based landmark co-registration between images. After co-registration, an overlay step is important for the integrated display of the data. Image fusion refers to a process of image co-registration followed by image overlay.

Image overlay involves visually merging two images into one display. For instance, a 2D real time ultrasound image is superimposed on a triplanar (3D) view of the initial image. Alternatively, for instance, a 3D ultrasound image is overlaid onto the initial image, by using a transparency overlay. A virtual ultrasound probe is then rendered at the top of the ultrasound image to provide a cue for the left-right orientation of the image relative to the physical ultrasound probe. A virtual ultrasound probe, as used herein, describes a digital representation of a physical ultrasound probe which is displayed by an ultrasound imaging modality. A physical ultrasound probe, as used herein, describes a portion of an ultrasound imaging system, which is moved by an operator in order to modify an image produced by the ultrasound imaging system. As the ultrasound probe is moved, the scene is re-rendered (e.g. at about 5 frames per second). The ultrasound image and initial image are often shown in different colors during image overlay in order to distinguish one from the other.

An alternative embodiment provides an alternative image fusion technique, which includes the following steps: generating an initial image and a corresponding ultrasound index image; generating an ultrasound image in real time; co-registering the index image with a real time image (e.g. using Philips Qlab software); and overlaying the initial image onto the real time image, using an image overlay algorithm. In this technique, co-registration involves a manual and/or image based initial image similarity assessment and an image based landmark co-registration between the index image and the real-time image.

An index image, as used herein, describes an ultrasound image that depicts a region of a patient body that is also imaged by an initial preoperative image. For instance, a CT imaging modality is used to generate an initial image of a region within a patient body, and a corresponding ultrasound index image is used to image a region having about equivalent size, shape, and/or location within the patient body.

In comparison to other methods of co-registration, the methods and systems provided herein have several advantages. The methods are non-invasive (compared to other methods that involve inserting artificial markers, for instance stainless steel beads, inside the body). The velocity vector field and/or displacement field account for a flexibility value of the target volume and/or surrounding structures, resulting in more accurate treatment of the target volume. Computation time is greatly reduced, due to (1) producing only one preoperative dataset, rather than several volumes corresponding to different phases of organ motion, (2) performing only one cross-modality image co-registration (e.g. CT to ultrasound or MRI to ultrasound), and (3) computing a velocity and/or displacement field using a single imaging modality (ultrasound), rather than multiple modalities.

The alternative image fusion technique has the following advantages: it avoids direct image co-registration between two imaging modalities; instead, it uses the index ultrasound image to indirectly match the initial (e.g. CT) image to the real-time ultrasound image; it does not require the use of artificial markers during the interventional treatment; the initial image could be gathered in advance of (e.g. a few days before) the interventional treatment. Moreover, if using an ultrasound imaging system with dual imaging capabilities, a CEUS initial image, and an ultrasound index image are obtained from the same imaging plane at the same time. Further, using an existing contrast image instead of a real time contrast image saves time and money and avoids imaging problems caused by a vapor cloud, which describes a collection of water vapor produced by thermally treating cells.

It will furthermore be apparent that other and further forms of the invention, and embodiments other than the specific and exemplary embodiments described above and in the claims, may be devised without departing from the spirit and scope of the appended claims and their equivalents, and therefore it is intended that the scope of this invention encompasses these equivalents and that the description and claims are intended to be exemplary and should not be construed as further limiting.

1. A method for computing non-invasively a velocity vector field of a flexible target volume within a bodily cavity, the method comprising:
generating a preoperative image of a region surrounding the target volume using a preoperative imaging modality, wherein the region comprises the target volume and wherein the preoperative image modality is not grayscale ultrasound, and producing a initial target volume calculation based upon the preoperative image;
generating an ultrasound image of a region surrounding the target volume using an ultrasound imaging modality, wherein the region comprises the target volume, spatially aligning the ultrasound image with the preoperative image using an image co-registration technique, thereby providing an updated target volume calculation based upon the spatially aligned ultrasound and preoperative images, and combining the ultrasound image with the preoperative image using an overlay technique; and
computing a velocity vector field of the target volume from spatially aligned ultrasound images of the region surrounding the target volume by comparing successive frames of ultrasound intensity data and using the velocity vector field to modify a target volume calculation in real time, wherein computing the velocity vector field is non-invasive and is adjusted to a flexibility value of the (i) target volume and (ii) surrounding tissue, thereby computing non-invasively a velocity vector field of a flexible target volume within a bodily cavity.

2. The method according to claim 1, wherein the preoperative image or preoperative modality is at least one selected from the group consisting of: magnetic resonance imaging, computed tomography, contrast enhanced ultrasound, and the like.

3. The method according to claim 1, wherein at least one of the initial target volume calculation and the updated target volume calculation further comprises at least one target volume parameter selected from the group consisting of: a location, an extent, and a shape of the target volume.

4. The method according to claim 1, wherein the ultrasonic image is a two-dimensional image.

5. The method according to claim 1, wherein the ultrasonic image is a three-dimensional image.

6. (canceled)

7. The method according to claim 1, wherein computing the velocity vector field further comprises computing a displacement field.

8. The method according to claim 7, wherein computing the velocity vector field and/or displacement field further comprises calculating at least one target volume parameter selected from the group consisting of: rotation, translation, and deformation of the target volume.

9. The method according to claim 1, further comprising reducing computation time by generating a single preoperative image, using a single image co-registration, and using a single imaging modality to compute the velocity vector field and/or a displacement field.

10. A method for guiding an interventional medical procedure for diagnosis or therapy of a flexible target volume, the method comprising:
using a velocity vector field and/or displacement field of the target volume to modify in real time a target volume calculation, wherein computing the corresponding field is non-invasive and adjusted to a flexibility value of the target volume and surrounding tissue, further wherein computing the velocity vector field includes (i) generating a preoperative image of a region surrounding the flexible target volume using a preoperative imaging modality, wherein the region comprises the flexible target volume and wherein the preoperative image modality is not grayscale ultrasound, and producing a initial target volume calculation based upon the preoperative image; (ii) generating an ultrasound image of a region surrounding the target volume using an ultrasound imaging modality, wherein the region comprises the target volume, spatially aligning the ultrasound image with the preoperative image using an image co-registration technique, thereby providing an updated target volume calculation based upon the spatially aligned ultrasound and preoperative images; and comparing successive frames of ultrasound intensity data from spatially aligned ultrasound images of the region surrounding the target volume;
generating at least one ultrasonic image of an interventional device in real time; and
using a real time ultrasonic image of the interventional device and the ultrasonic target volume calculation to alter the placement of the interventional device, thereby guiding an interventional medical procedure for diagnosis or therapy of a flexible target volume.

11. The method according to claim 10, wherein the target volume calculation further comprises at least one target volume parameter selected from the group consisting of: a location, an extent, and a shape of the target volume.

12. The method according to claim 10, wherein the ultrasonic image is a two-dimensional image.

13. The method according to claim 10, wherein the ultrasonic image is a three-dimensional image.

14. A method for combining a plurality of types of medical images for guiding an interventional medical procedure, the method comprising:
generating a initial image of a region surrounding a flexible target volume using an imaging modality, wherein the region comprises the flexible target volume and wherein the modality is not grayscale ultrasound, and generating a corresponding ultrasound index image, wherein generating the initial image further includes producing a initial target volume calculation based upon the initial image;
generating in real time an ultrasound image of the flexible target volume, and making an image-based co-registration between the ultrasound index image and a real time ultrasound image to indirectly match the initial image to the real time ultrasound image and spatially align the real time ultrasound image with the initial image, thereby providing an updated target volume calculation based upon the spatially aligned images; and
combining the initial image with the real time ultrasound image, using at least one of an overlay technique and the ultrasound index image, wherein combining includes computing a velocity vector field of the flexible target volume from spatially aligned ultrasound images of the region surrounding the flexible target volume by comparing successive frames of ultrasound intensity data and using the velocity vector field to modify a target volume calculation in real time, wherein computing the velocity vector field is non-invasive and is adjusted to a flexibility value of the (i) target volume and (ii) surrounding tissue.

15. The method according to claim 14, wherein the image or the imaging modality is at least one selected from the group consisting of: computed tomography, magnetic resonance imaging, contrast enhanced ultrasound, and the like.

16. The method according to claim 14, wherein the real time ultrasound image is generated during the interventional procedure.

17. A system for guiding an interventional medical procedure for diagnosis or therapy of a flexible target volume using a plurality of imaging modalities, comprising:
a preoperative imaging modality for generating a preoperative image of a region surrounding the target volume and for producing a initial target volume calculation based upon the preoperative image, wherein the preoperative imaging modality is not grayscale ultrasound;
an ultrasound imaging modality for generating in real time an image of an interventional medical device in a region surrounding the target volume, spatially aligning the ultrasound image with the preoperative image, and computing a velocity vector field and/or displacement field of the target volume, wherein the corresponding field is used for generating an updated target volume calculation based upon the spatially aligned ultrasound and preoperative images, wherein computing further comprises comparing successive frames of ultrasound intensity data and using the velocity vector field and/or displacement field to modify the updated target volume calculation in real time; and
the interventional medical device for inserting into the target volume, wherein the updated target volume calculation and the real time image of the interventional device are used to alter the placement of the interventional device.

18. The system according to claim 17, wherein the preoperative modality or preoperative image is at least one selected from the group consisting of: computed tomography, magnetic resonance imaging, contrast enhanced ultrasound, and the like.

19. The method according to claim 18, wherein at least one of the initial target volume calculation and the updated target volume calculation further comprises at least one target volume parameter selected from the group consisting of: a location, an extent, and a shape of the target volume.
